(19) United States
(12) Patent Application Publication
(10) Pub. No.: US 2006/0160080 A1
(43) Pub. Date: Jul. 20, 2006

(54) Avian sex determination method

(75) Inventor: Michael Clinton, Midlothian (GB)

Correspondence Address: Wilmer Cutler Pickering Hale & Dorr, 60 State Street, Boston, MA, 02109, US

(73) Assignee: Roslin Institute (Edinburgh), Roslin, Midlothian (GB)

(21) Appl. No.: 10/524,475

(22) PCT Filed: Aug. 13, 2003
(86) PCT No.: PCT/GB03/03536

§ 371 Date: Jan. 20, 2006

(30) Foreign Application Priority Data
Aug. 14, 2002 (GB) 0218955.3

(51) Int. Cl. C12Q 1/68

(52) U.S. Cl. 435/6

(57) ABSTRACT

A method for the determination of the sex of an avian subject is provided which comprises analysis of a sample from said subject with a nucleic acid probe comprising an at least 6 base pair fragment from a Female Associated Factor (FAF) nucleic acid sequence, or with an antibody to a protein coded for by said sequences.

The present invention relates to a method for sexing individual subjects of avian species.

Adults and particularly offspring of many avian species are monomorphic, making determination of sex difficult. Nucleic acid probes that hybridise to the DNA of the female-specific W chromosome have lead to molecular solutions to this problem for some species. However, use of such techniques has proved to be difficult and in many cases, their taxonomic range is limited. More recently, polymerase chain reaction (PCR) based approaches that are technically simpler and that have broader taxonomic utility have been developed.

Sex identification methods have also been based upon examining differences in intron size between the female W specific chromosome and the Z chromosome, which occurs in both sexes (female, ZW; male, ZZ) (Ellegren 1996, Kahn et al 1998). Another approach has been to identify specific genes located on the W chromosome. Analysis of the chromobox-helicase-DNA-binding gene (CHD) shows that it contains sequences specific to the W chromosome and can be used for determination of the sex of most birds (Griffiths, et al 1998). A combination of the analysis of intron size difference between sexes and the chromosome specific CHD gene has also been proposed (Fridolfsson, A-K and Ellegren, H, 1999). Another method proposed has been to analyse W-specific repeat sequences in order to determine the sex of chick embryos (Clinton, 1994). However, this method required separate sexing and control PCR reactions. A rapid and simple single tube chicken sexing protocol based on a PCR analysis of W chromosome specific sequences has been devised more recently (Clinton et al 2001). Other methods have been proposed in WO 96/39505 based on an analysis of DNA sequences (introns and exons) encoding two genes located on the Z and W chromosomes of birds. Other means for analysis have been proposed in U.S. Pat. No. 5,679,514 and U.S. Pat. No. 5,707,809.

Such methods of sex determination generally require technical expertise and specialist facilities, and are not susceptible to ready automation, especially in agricultural environments. The sequences referred to also have variants (homologues in the case of genes) of that sequence present on the Z-chromosome (i.e. the avian sex chromosome that is found in both sexes).

The avian gene WPKCI has been shown to be conserved widely on the avian W chromosome and expressed actively in the female chicken embryo before the onset of gonadal differentiation. It is suggested that WPKCI may play a role in the differentiation of the female gonad by interfering with the function of PKCI or by exhibiting its unique function in the nucleus (Hori et al 2000). This gene has also been identified as ASW (avian sex-specific W-linked) (O'Neill et al 2000).

Accurate determination of the sex of avians is a particularly important issue for the poultry industry for both economic and welfare reasons. Companies which produce egg-layer strains of chickens (“layers”) would prefer to be able to (inexpensively) determine the sex of birds at hatch and just raise the females; companies which produce meat strains of chickens (“broilers”) would prefer to (inexpensively) sex birds at hatch and just raise males as they grow much faster and eat less. Currently, most “broiler” producers accept the inefficiency of producing and rearing female birds, whilst a proportion of the “layer” producers use relatively expensive procedures to determine the sex of one-day old chicks.

The use of sex determination procedures on one-day old chicks has significant welfare implications, in particular the disposal of unwanted male chicks from layer strains of chickens. Recent estimates suggest that at least 280 million such chicks have to be disposed of each year in the European Union alone. The means of disposal used in practice are killing the chicks by maceration, or by gassing or by electrocution (both of the latter followed by incineration). The use of maceration has been recommended as the other can techniques leave approximately 40% of the chicks alive prior to incineration.

There exists a need, therefore, for simple, accurate methods that can be used at poultry farms that overcome the problems in the prior art and allow for improved animal welfare.

It has now been surprisingly discovered that a W-chromosome specific transcript can be used as the basis for a sex determination method that overcomes the problems previously encountered in this field to date. The W-specific transcript is surprising as it is 3′ to 5′ in relation to the transcribed strand (5′ to 3′) for the gene WPKCI already known and there is no known Z-chromosome copy.

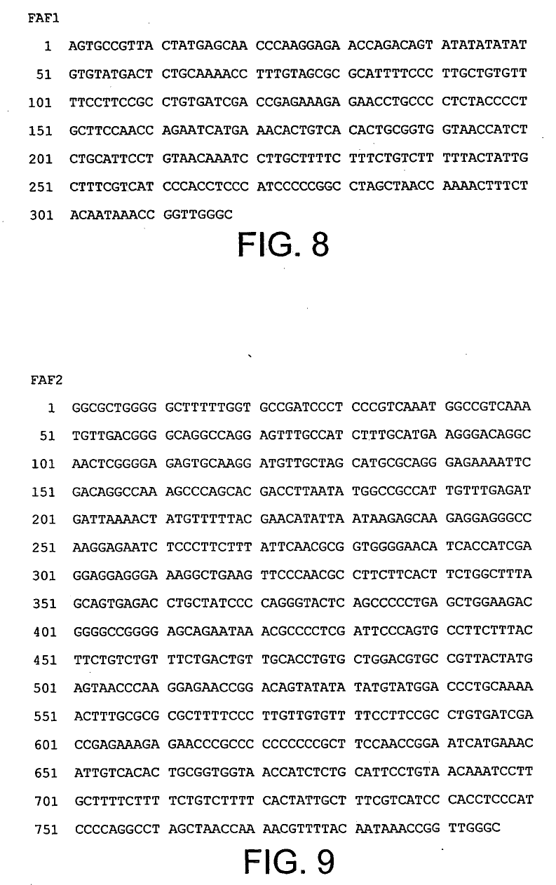

According to a first aspect of the invention, there is provided a method for the determination of the sex of an avian subject, the method comprising contacting a sample from said subject with a nucleic acid probe comprising an at least 6 base pair fragment from a target nucleic acid sequence as shown in FIGS. 8 to 14, or a sequence complementary or homologous thereto.

The present invention provides methods for the determination of the sex of an avian subject, i.e. whether the subject is male or female. The methods of the present invention can be used to determine the sex of a subject of the Class Aves, for example bird species of agricultural importance such as Gallus gallus (chicken), turkeys, quail, guinea fowl, commonly referred to as poultry. Such methods may find application in relation to other bird species, such as those bred in captivity, kept as domestic pets, or kept in zoological institutions and examples include penguins, parrots, and rare bird species threatened with extinction, and/or the subjects of breeding programmes for conservation. The subject avian being analysed may be an embryo, a newly hatched chick or a mature adult bird.

Samples that can be assayed according to a method of the present invention include but are not limited to samples of allantois or amnion from the egg, i.e. allantoic fluid or amniotic fluid, of an avian subject containing a developing embryo; other sources of suitable samples include any convenient sample of a biological nature containing cells, tissue or organs, for example, muscle, heart, brain, lung, liver, chorioallantoic membrane, mesonephrous and blood.

Such methods can be carried out on samples removed from an egg without compromising the viability of the egg using standard procedures. Samples may be removed from the egg manually or by using an automated approach. Machines intended for delivery of vaccine to eggs for incubation can be altered to sample the fluids of the egg in the same manner. The methods can, of course, be performed equally on cultured cells in vitro.

The samples to be analysed according to a method of the present invention, may be analysed by means of a DNA amplification procedure, such as the polymerase chain reaction (PCR), or by means of RNA analysis, for example Northern blot, or a Southern blot (Sambrook, J., & Russell, D. W., “Molecular Cloning”, Cold Spring Harbor Laboratory Press (2001)), or by using an Invader® RNA Assay (Third Wave—www.twt.com). Such methods are based on the hybridisation of a probe (or primer) nucleic acid sequence to the target nucleic acid of interest in a sample.

The Invader® assay is based on a “perfect-match” enzyme-substrate reaction. Certain endonuclease enzymes are used which recognise and cut only the specific structure formed during the Invader® process. The method relies upon linear amplification of the signal generated, rather than on exponential amplification of the target as in PCR-based approaches. This allows for easy quantification of the target concentration and reduces the effects of sample contamination which may result from exponential target amplification. The system is applicable to analysis of RNA and DNA samples. In the Invader® process, two short DNA probes hybridise to the target to form the structure recognised by the endonuclease enzymes. The enzyme then cuts one of the probes to release a short DNA sequence. Each target can induce the release of several thousand such sequence fragments per hour. Each released sequence fragment can bind to a fluorescently-labelled probe and form another cleavage structure. When the endonuclease cuts the labelled probe, the probe emits a detectable fluorescent signal. Each released DNA sequence fragment can generate thousands of signals per hour, yielding millions of detectable signals per target (Heisler, L. M. & Lonergan, S. C., Biomol. Eng. in press, (2001); Fors et al Pharmacogenomics, 1, 219-229 (2000); Heisler et al Clinical Hemostasis Review, 14 (11) 10-11 (2000); Leider, K. W. Advance for Laboratory Managers 70-71 (February 2000); Treble et al Gene and Medicine 4 68-72 (2000); Leider, K. W. Advance for Laboratory Managers 50-52 (November 1999)).

One of the advantages of an Invader® RNA Assay is that it could be carried out in a farm location without complicated equipment or sensitive materials and having no need for specialist experience once initial training has been provided. For example, an Invader® RNA Assay may be devised based upon the 324 bp FAF fragment. This could rely on the genomic DNA sequence or on the RNA transcript. The constituents of the assay can be provided dried down, in a multi-well format, such as a 96-well or 384-well plate. In use the currently available Invader® RNA Assay would comprise a probe/Invader® mix (a FAF probe in the present invention), a signal probe, a signal buffer, Cleavase VIII enzyme, a “no target” control, and Rnase-free water. The biological sample (DNA, RNA, amniotic fluid, allantoic fluid, lysed tissue, or lysed blood) can then be added to the wells. The plate can then be incubated in a standard water bath for a defined period and then scanned in a fluorescence reader. The means for detecting the florescence may by a fluorescence resonance energy transfer (FRET) assay.

An RNA-based Invader® assay may be particularly advantageous given the anti-sense nature of the FAF sequence. An RNA Invader® assay would have an oligonucleotide complementary to the transcribed region so would not bind to anything transcribed of the other complementary DNA strand.

The probe to be used in the methods of the invention can be designed according to the general principles of the assay system used. The length of the probe used will depend on whether the assay system is PCR, Northern blot, Southern blot, or an Invader® assay. The probe sequence is at least 6 base pairs (bp) in length and can be any 6 bp sequence from a nucleic acid sequence as shown in FIGS. 8 to 14 or a complementary sequence thereto, as appropriate with respect to the assay system used. The sequences of FIGS. 8 to 14 encode a female specific RNA transcript and can be referred to as “Female Associated Factor” or FAF. Such sequences are therefore target sequences.

The probe sequence can be from 6 to 10 bp, at least 10 bp, or at least 15 bp, 10 bp to 15 bp, 15 bp to 20 bp, at least 20 bp, 20 bp to 25 bp and so on up to at least 324 bp. Additional nucleotide residues can be included in the probes designed as required provided that no disruption to the binding of probe to target is seen.

So for a Northern or a Southern assay method, a full-length cDNA probe can be used, or fragments or oligonucleotides based on the full length sequence. For an Invader® assay, probe lengths of from 15 base pairs up to a full length cDNA could be used based on any one of the sequences shown in FIGS. 8 to 14.

The nucleic acid may be deoxyribonucleic acid (DNA) or ribonucleic acid (RNA). Suitably, the nucleic acid is an isolated nucleic acid.

Methods of the present invention can determine whether an individual avian subject is male by virtue of the absence of female-specific RNA transcript or DNA sequence, or whether the subject is female by virtue of the presence of the female-specific RNA transcript or DNA sequence in the sample, in which the female-specific RNA transcript, or DNA sequence is derived from a sequence of FIGS. 8 to 14, or a sequence complementary or homologous thereto.

The percent identity of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA (1990) 87:2264-2268, modified as in Karlin and Altschul (1993) Proc. Natl. Acad. Sci. USA 90:5873-5877. The NBLAST and XBLAST programs of Altschul et al, J. Mol. Biol. (1990) 215:403-410 have incorporated such an algorithm. BLAST nucleotide searches can be performed with the NBLAST program, score=100, wordlength=12 to obtain nucleotide sequences homologous to a nucleic acid molecules of the invention. To obtain gapped alignments for comparison purposes, Gapped BLAST can be utilised as described in Altschul et al, Nucleic Acids Res. (1997) 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., NBLAST) can be used. See www.ncbi.nlm.nih.gov.

Another example of a mathematical algorithm utilised for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti Comput. Appl. Biosci. (1994) 10:3-5; and PASTA described in Pearson and Lipman Proc. Natl. Acad. Sci. USA (1988) 85:2444-8. Within FASTA, ktup is a control option that sets the sensitivity and speed of the search.

A nucleic acid sequence which is complementary to a nucleic acid sequence useful in a method of the present invention is a sequence which hybridises to such a sequence under stringent conditions, or a nucleic acid sequence which is homologous to or would hybridise under stringent conditions to such a sequence but for the degeneracy of the genetic code, or an oligonucleotide sequence specific for any such sequence. The nucleic acid sequences include oligonucleotides composed of nucleotides and also those composed of peptide nucleic acids. Where the nucleic sequence is based on a fragment of the sequences of the invention, the fragment may be at least any ten consecutive nucleotides from the gene, or for example an oligonucleotide composed of from 20, 30, 40, or 50 nucleotides.

Stringent conditions of hybridisation may be characterised by low salt concentrations or high temperature conditions. For example, highly stringent conditions can be defined as being hybridisation to DNA bound to a solid support in 0.5M NaHPO₄, 7% sodium dodecyl sulfate (SDS), 1 mM EDTA at 65° C., and washing in 0.1×SSC/0.1% SDS at 68° C. (Ausubel et al eds. “Current Protocols in Molecular Biology” 1, page 2.10.3, published by Green Publishing Associates, Inc. and John Wiley & Sons, Inc., New York, (1989)). In some circumstances less stringent conditions may be required. As used in the present application, moderately stringent conditions can be defined as comprising washing in 0.2×SSC/0.1% SDS at 42° C. (Ausubel et al (1989) supra). Hybridisation can also be made more stringent by the addition of increasing amounts of formamide to destabilise the hybrid nucleic acid duplex. Thus particular hybridisation conditions can readily be manipulated, and will generally be selected according to the desired results. In general, convenient hybridisation temperatures in the presence of 50% formamide are 42° C. for a probe which is 95 to 100% homologous to the target DNA, 37° C. for 90 to 95% homology, and 32° C. for 70 to 90% homology.

Examples of preferred nucleic acid sequences for use in a method of the present invention are the sequences of the invention shown in FIGS. 8 to 14. Complementary or homologous sequences may be 75%, 80%, 85%, 90%, 95%, 99% similar to such sequences.

The advantages of the methods of the present invention are that the sex of an avian subject can be readily and easily determined based on a single biological sample. There are immediate animal welfare implications in agriculture as the previous practice of whole chick homogenisation can be discontinued.

In a preferred embodiment of the invention there is provided a method for determining the sex of an avian subject, the method comprising the steps of:
(1) obtaining a suitable sample from an avian subject being
   (i) an avian embryo in ovo; or
   (ii) an individual avian
(2) preparing sample for analysis;
(3) probing the sample with a nucleic acid probe based on a sequence of FIGS. 8 to 14; and
(4) analysing the results of step (3) to determine if individual is male or female.

In a further preferred embodiment of the invention, there is provided a method for determining the sex of an avian embryo, the method comprising the steps of:
(1) obtaining a suitable sample from an avian egg
(2) preparing sample for analysis;
(3) probing the sample with a nucleic acid probe based on a sequence of FIGS. 8 to 14; and
(4) analysing the results of step (3) to determine if individual is male or female.

In certain embodiments of the invention, the use of the Invader® assay may be preferable. It may also be advantageous to analyse RNA transcripts present in the sample using the Invader® assay. Alternatively, the Polymerase Chain Reaction (PCR) may be used, either standard PCR and gel analysis, or a quantitative PCR analysis such as Taqman®.

According to a second aspect of the invention, there is provided the use of a nucleic acid sequence or a fragment thereof according to any one of FIGS. 8 to 14 in a method according to the first aspect of the invention.

According to a third aspect of the invention there is provided a nucleic acid sequence as shown in any one of FIGS. 8 to 14. Such isolated sequences have use in methods and uses in accordance with the first and second aspects of the invention in determining the sex of an avian subject.

According to a fourth aspect of the invention there is provided a kit of parts comprising a nucleic acid probe comprising an at least 6 base pair fragment from a nucleic acid sequence as shown in any one of FIGS. 8 to 14 for determining the sex of an avian subject or a sequence complementary or homologous thereto. The kit may further comprise instructions for use according to a method of the invention. The probe may suitably be provided in a removably sealed container.

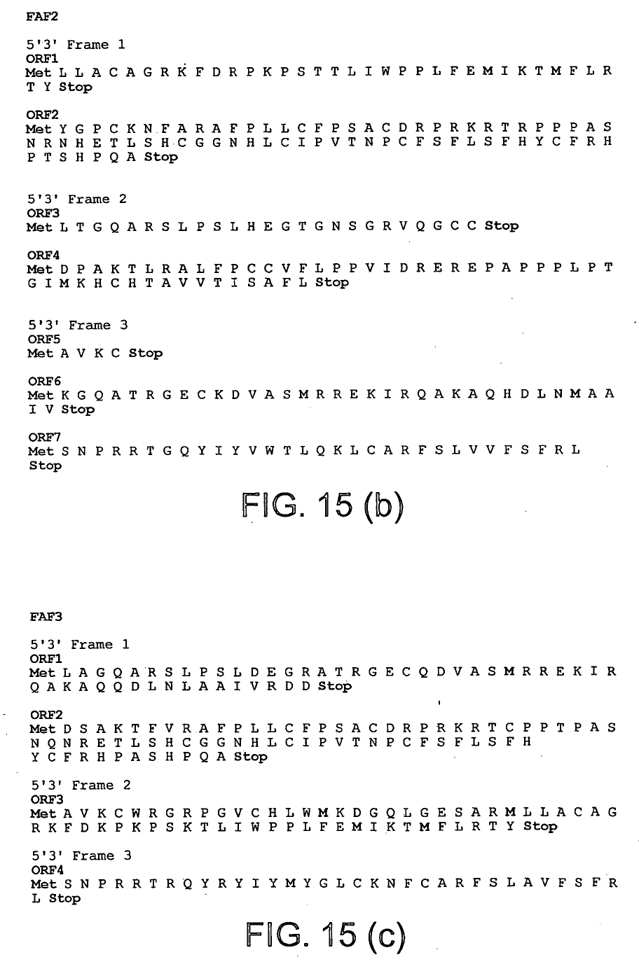

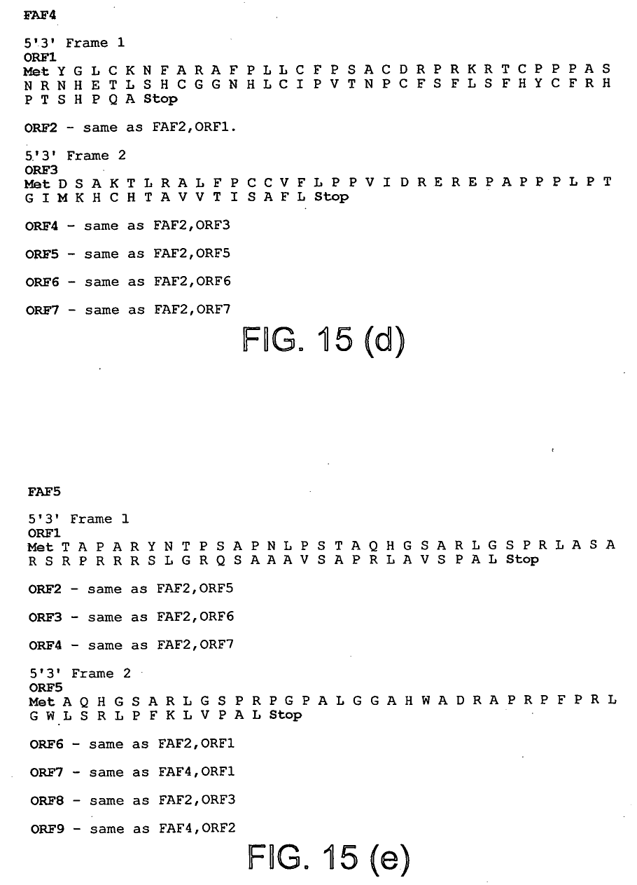

According to a fifth aspect of the invention, there is provided a polypeptide or fragment thereof coded for by a nucleic acid sequence of any one of FIGS. 8 to 12. The term “polypeptide” includes both peptide and protein, unless the context specifies otherwise. Examples of such peptide sequences are shown in FIG. 15.

Such polypeptides include analogues, homologues, orthologues, isoforms, derivatives, fusion proteins and proteins with a similar structure or are a related polypeptide as herein defined.

The term “analogue” as used herein refers to a polypeptide that possesses a similar or identical function as a protein coded for by a nucleic acid sequence of the invention but need not necessarily comprise an amino acid sequence that is similar or identical to an amino acid sequence of the invention, or possess a structure that is similar or identical to that of protein of the invention. As used herein, an amino acid sequence of a polypeptide is “similar” to that of a polypeptide of the invention if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of a polypeptide of the present invention; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of a polypeptide sequence of the invention; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding a polypeptide of the invention.

As used herein, a polypeptide with “similar structure” to that of a polypeptide of the invention refers to a polypeptide that has a similar secondary, tertiary or quaternary structure as that of a polypeptide of the invention. The structure of a polypeptide can determined by methods known to those skilled in the art, including but not limited to, X-ray crystallography, nuclear magnetic resonance, and crystallographic electron microscopy.

The term “fusion protein” as used herein refers to a polypeptide that comprises (i) an amino acid sequence of a polypeptide of the invention, a fragment thereof, a related polypeptide or a fragment thereof and (ii) an amino acid sequence of a heterologous polypeptide (i.e., not a polypeptide sequence of the present invention).

The term “homologue” as used herein refers to a polypeptide that comprises an amino acid sequence similar to that of a protein of the invention but does not necessarily possess a similar or identical function.

The term “orthologue” as used herein refers to a non-human polypeptide that (i) comprises an amino acid sequence similar to that of a protein of the invention and (ii) possesses a similar or identical function.

The term “related polypeptide” as used herein refers to a homologue, an analogue, an isoform of, an orthologue, or any combination thereof of a protein of the invention.

The term “derivative” as used herein refers to a polypeptide that comprises an amino acid sequence of a polypeptide of the invention which has been altered by the introduction of amino acid residue substitutions, deletions or additions. The derivative polypeptide possess a similar or identical function as polypeptides of the invention.

The term “fragment” as used herein refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues) of the amino acid sequence of a polypeptide of the invention. The fragment of may or may not possess a functional activity of such polypeptides.

The term “isoform” as used herein refers to variants of a polypeptide that are encoded by the same gene, but that differ in their isoelectric point (pI) or molecular weight (MW), or both. Such isoforms can differ in their amino acid composition (e.g. as a result of alternative splicing or limited proteolysis) and in addition, or in the alternative, may arise from differential post-translational modification (e.g., glycosylation, acylation, phosphorylation). As used herein, the term “isoform” also refers to a protein that exists in only a single form, i.e., it is not expressed as several variants.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA (1990) 87:2264-2268, modified as in Karlin and Altschul (1993) Proc. Natl. Acad. Sci. USA 90:5873-5877. The NBLAST and XBLAST programs of Altschul et al, J. Mol. Biol. (1990) 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilised as described in Altschul et al, Nucleic Acids Res. (1997) 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilised for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti Comput. Appl. Biosci. (1994) 10:3-5; and FASTA described in Pearson and Lipman Proc. Natl. Acad. Sci. USA (1988) 85:2444-8. Within FASTA, ktup is a control option that sets the sensitivity and speed of the search.

The skilled person is aware that various amino acids have similar properties. One or more such amino acids of a substance can often be substituted by one or more other such amino acids without eliminating a desired activity of that substance. Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). Substitutions of this nature are often referred to as “conservative” or “semi-conservative” amino acid substitutions.

Amino acid deletions or insertions may also be made relative to the amino acid sequence of a polypeptide sequence of the invention. Thus, for example, amino acids which do not have a substantial effect on the activity of such polypeptides, or at least which do not eliminate such activity, may be deleted. Amino acid insertions relative to the sequence of polypeptides of the invention can also be made. This may be done to alter the properties of a protein of the present invention (e.g. to assist in identification, purification or expression, where the protein is obtained from a recombinant source, including a fusion protein. Such amino acid changes relative to the sequence of a polypeptide of the invention from a recombinant source can be made using any suitable technique e.g. by using site directed mutagenesis. The molecule may, of course, be prepared by standard chemical synthetic techniques, e.g. solid phase peptide synthesis, or by available biochemical techniques.

It should be appreciated that amino acid substitutions or insertions within the scope of the present invention can be made using naturally occurring or non-naturally occurring amino acids. Whether or not natural or synthetic amino acids are used, it is preferred that only L-amino acids are present.

According to a sixth aspect of the invention, there is provided a vector comprising a nucleic acid sequence of any one FIGS. 8 to 12. The term “vector” or “expression vector” generally refers to any nucleic acid vector which may be RNA, DNA or cDNA.

The term “expression vector” may include, among others, chromosomal, episomal, and virus-derived vectors, for example, vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. Generally, any vector suitable to maintain, propogate or express nucleic acid to express a polypeptide in a host may be used for expression in this regard.

In certain embodiments of the invention, the vectors may provide for specific expression. Such specific expression may be inducible expression or expression only in certain types of cells or both inducible and cell-specific. Preferred among inducible vectors are vectors that can be induced for expression by environmental factors that are easy to manipulate, such as temperature and nutrient additives. Particularly preferred among inducible vectors are vectors that can be induced for expression by changes in the levels of chemicals, for example, chemical additives such as antibiotics. A variety of vectors suitable for use in the invention, including constitutive and inducible expression vectors for use in prokaryotic and eukaryotic hosts, are well known and employed routinely by those skilled in the art.

Recombinant expression vectors will include, for example, origins of replication, a promoter preferably derived from a highly expressed gene to direct transcription of a structural sequence, and a selectable marker to permit isolation of vector containing cells after exposure to the vector.

Expression vectors may comprise an origin of replication, a suitable promoter and enhancer, and also any necessary ribosome binding sites, polyadenylation regions, splice donor and acceptor sites, transcriptional termination sequences, and 5′-flanking non-transcribed sequences that are necessary for expression. Preferred expression vectors according to the present invention may be devoid of enhancer elements.

The promoter sequence may be any suitable known promoter, for example the human cytomegalovirus (CMV) promoter, the CMV immediate early promoter, the HSV thymidine kinase promoter, the early and late SV40 promoters or the promoters of retroviral LTR's, such as those of the Rous sarcoma virus (“RSV”), and metallothionein promoters, such as the mouse metallothionein-I promoter. The promoter may comprise the minimum sequence required for promoter activity (such as a TATA box without enhancer elements), for example, the minimal sequence of the CMV promoter (mCMV).

The expression vectors may also include selectable markers, such as antibiotic resistance, which enable the vectors to be propagated.

The nucleic acid sequence contained in the expression vector of this aspect of the invention may be a reporter transcription unit lacking a promoter region, such as a chloramphenicol acetyl transferase (“CAT”) transcription unit. As is well known, introduction into an expression vector of a promoter-containing fragment at a restriction site upstream of the CAT gene engenders the production of CAT activity, which can be detected by standard CAT assays. The application of reporter genes relates to the phenotype of these genes which can be assayed in a transformed organism and which is used, for example, to analyse the induction and/or repression of gene expression. Reporter genes for use in studies of gene regulation include other well known reporter genes including the lux gene encoding luciferase which can be assayed by a bioluminescence assay, the uidA gene encoding β-glucuronidase which can be assayed by a histochemical test, the aphIV gene encoding hygromycin phosphotransferase which can be assayed by testing for hygromycin resistance in the transformed organism, the dhfr gene encoding dihydrofolate reductase which can be assayed by testing for methotrexate resistance in the transformed organism, the neo gene encoding neomycin phosphotransferase which can be assayed by testing for kanamycin resistance in the transformed organism and the lacZ gene encoding β-galactosidase which can be assayed by a histochemical test. All of these reporter genes are obtainable from E. coli except for the lux gene. Sources of the lux gene include the luminescent bacteria Vibrio harveyii and V. fischeri, the firefly Photinus pyralis and the marine organism Renilla reniformis.

According to a seventh aspect of the invention, there is provided a host cell comprising a vector as described above. Suitably, the host cell is stably transfected by the vector. The nucleic acid sequence of the invention may be incorporated into the genome of the cell or it may be expressed episomally. In general, the host cell will be of an avian species as described herein, but other host cells such as bacteria or yeast are included within the scope of this aspect of the invention.

Introduction of an expression vector into the host cell can be effected by calcium phosphate transfection, DEAE-dextran mediated transfection, microinjection, cationic lipid-mediated transfection, electroporation, transduction, scrape loading, ballistic introduction, infection of other methods. Such methods are described in many standard laboratory manuals, such as Sambrook et al., Molecular Cloning: A Laboratory Manual, 2^{nd} Ed., Cold Spring Harbor Laboratory Press, Cold Spring Harbor, N.Y. (1989).

According to an eighth aspect of the invention, there is provided an antibody to a polypeptide of the fifth aspect of the invention.

The antibody may be a polyclonal antibody or a monoclonal antibody. Polyclonal antibodies can be raised by stimulating their production in a suitable animal host (e.g. a mouse, rat, guinea pig, rabbit, sheep, chicken, goat or monkey) when the substance of the present invention is injected into the animal. If necessary an adjuvant may be administered together with the substance of the present invention. The antibodies can then be purified by virtue of their binding to a protein of the invention or as described further below. Monoclonal antibodies can be produced from hybridomas. These can be formed by fusing myeloma cells and spleen cells which produce the desired antibody in order to form an immortal cell line. This is the well known Kohler & Milstein technique (Nature 256 52-55 (1975)).

Techniques for producing monoclonal and polyclonal antibodies which bind to a particular protein are now well developed in the art. They are discussed in standard immunology textbooks, for example in Roitt et al, Immunology second edition (1989), Churchill Livingstone, London.

In addition to whole antibodies, the present invention includes derivatives thereof which are capable of binding to a polypeptide of the invention. Thus the present invention includes antibody fragments and synthetic constructs. Examples of antibody fragments and synthetic constructs are given by Dougall et al in Tibtech 12 372-379 (September 1994). Antibody fragments include, for example, Fab, F(ab′)₂ and Fv fragments (see Roitt et al [supra]). Fv fragments can be modified to produce a synthetic construct known as a single chain Fv (scFv) molecule. This includes a peptide linker covalently joining Vₕ and Vₗ regions which contribute to the stability of the molecule.

Other synthetic constructs include CDR peptides. These are synthetic peptides comprising antigen binding determinants. Peptide mimetics may also be used. These molecules are usually conformationally restricted organic rings which mimic the structure of a CDR loop and which include antigen-interactive side chains. Synthetic constructs also include chimaeric molecules. Thus, for example, humanised (or primatised) antibodies or derivatives thereof are within the scope of the present invention. An example of a humanised antibody is an antibody having human framework regions, but rodent hypervariable regions. Synthetic constructs also include molecules comprising a covalently linked moiety which provides the molecule with some desirable property in addition to antigen binding. For example the moiety may be a label (e.g. a detectable label, such as a fluorescent or radioactive label) or a pharmaceutically active agent.

The antibodies or derivatives thereof specific for a polypeptide of the invention have a variety of other uses. They can be used in purification and/or identification of such proteins or a cell that expresses the polypeptides. As a result they may be used in a diagnostic method according to the present invention.

After the preparation of a suitable antibody to a protein of the invention, it may be isolated or purified by one of several techniques commonly available (for example, as described in Antibodies: A Laboratory Manual, Harlow and Lane, eds. Cold Spring Harbor Laboratory Press (1988)). Generally suitable techniques include peptide or protein affinity columns, HPLC or RP-HPLC, purification on Protein A or Protein G columns, or combinations of these techniques. Recombinant antibodies to polypeptides of the invention can be prepared according to standard methods, and assayed for specificity for these proteins using procedures generally available, including ELISA, ABC, dot-blot assays etc.

According to a ninth aspect of the invention, there is provided a method for the determination of the sex of an avian subject, the method comprising contacting a sample from said subject with an antibody to a polypeptide of the fifth aspect of the invention. Suitably the antibody is detectably labelled or is itself contacted by a reporter antibody that is detectably labelled. For example the label may be a fluorescent or radioactive label. Alternatively, the antibody may be detected by an anti-idiotype antibody which is labelled.

According to a tenth aspect of the invention, there is provided a kit of parts comprising an antibody as defined above for determining the sex of an avian subject. Suitably, the antibody is supplied in a removably sealed container. The kit may further comprise instructions for use according to a method of the invention.

Preferred features for the second and subsequent aspects of the invention are as for the first aspect mutatis mutandis.

The invention will now be further described by way of reference to the following Examples and Figures which are provided for the purposes of illustration only and are not to be construed as being limiting on the invention. Reference is made to a number of Figures in which:

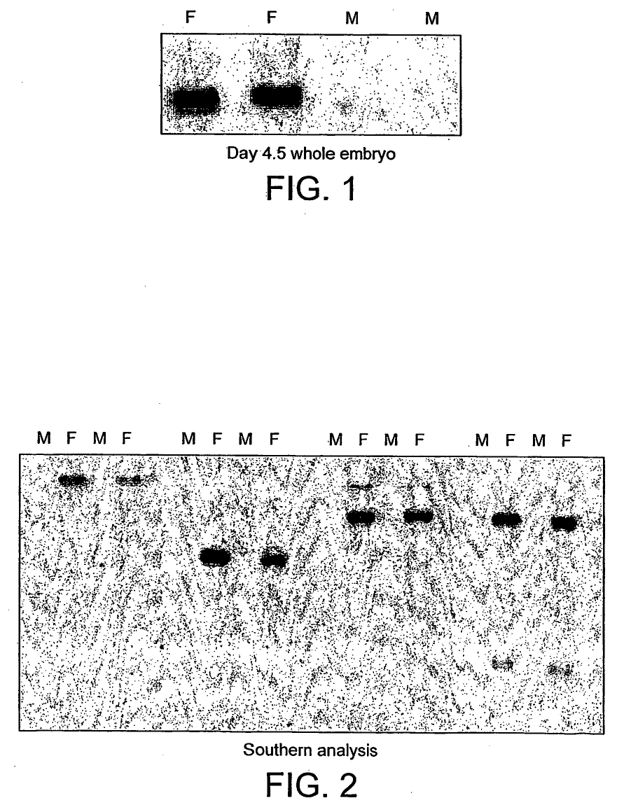

FIG. 1 shows the results of Northern analysis of RNA samples from a day 4.5 whole chick embryos using differential display clone 378.2.6 as a probe. Two major bands were detected in females and transcript sizes were approximately 800 bp and 1300 bp.

FIG. 2 shows the results of a Southern blot of male and female chicken genomic DNA digested with four different restriction enzymes probed with ³²P-labelled cDNA clone 378.2.6.

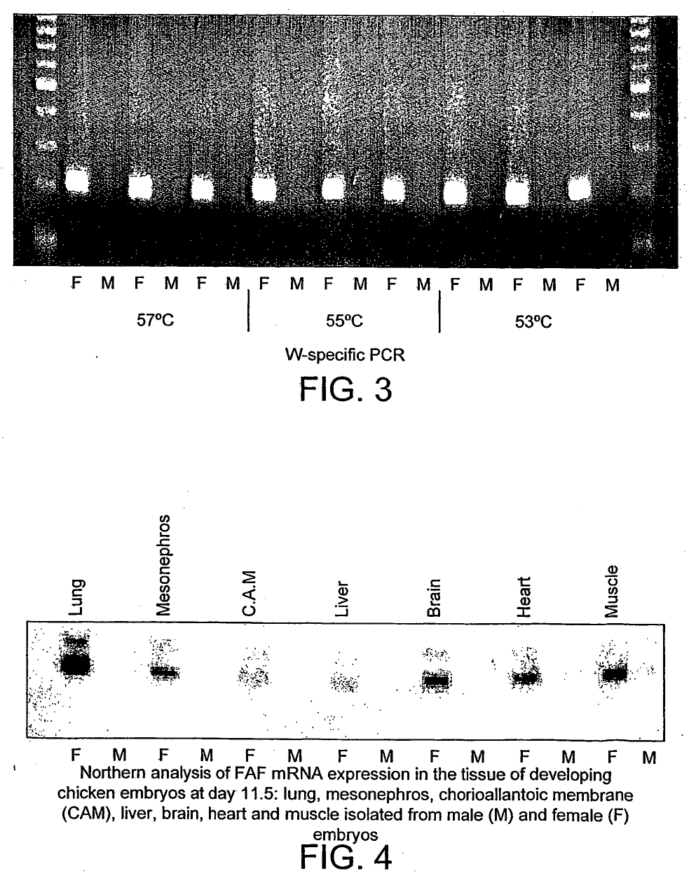

FIG. 3 shows the results of W-specific PCR at 57° C., 55° C. and 53° C. using PCR primers designed from the 796 bp sequence of FAF 4.

FIG. 4 shows the results of a northern blot of total RNA from male and female tissues—heart, brain, lung, liver, chorioallantoic membrane and mesonephrous-probed with ³²P-labelled clone 378.2.6.

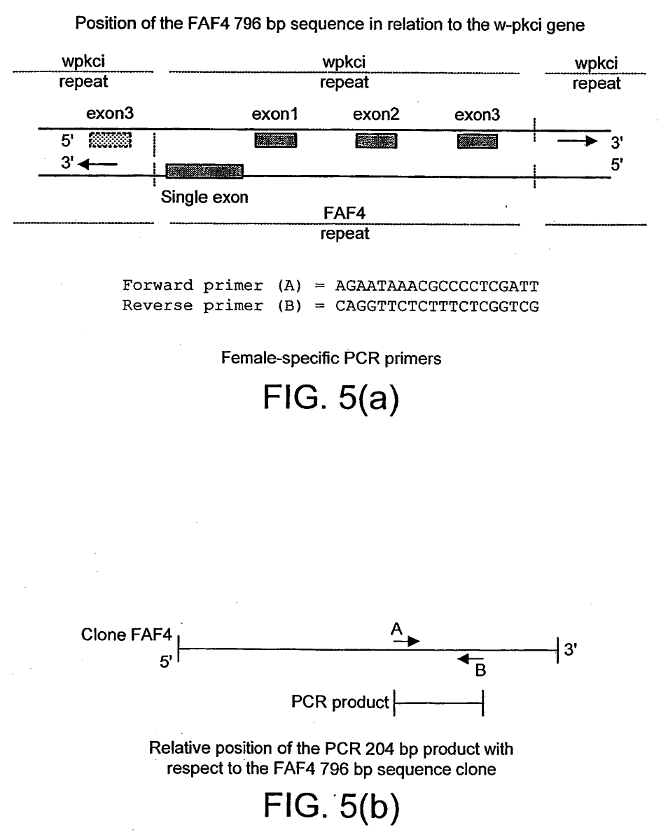

FIG. 5(a) shows the position of the FAF-4 796 bp sequence in relation to the w-pkci gene. FIG. 5(b) shows the relative position of the PCR 204 bp product with respect to the FAF-4 796 bp sequence clone.

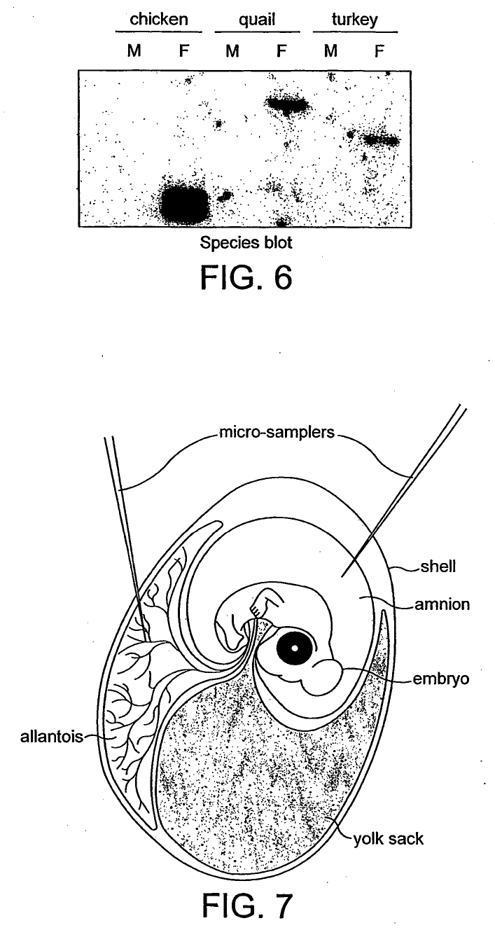

FIG. 6 shows a species blot probed with FAF display fragment for chicken, quail and turkey.

FIG. 7 shows a diagrammatical representation of sampling of amnion and allantois of a developing chick embryo.

FIG. 8 shows the nucleotide sequence for the differential display fragment of 324 bp (FAF-1).

FIG. 9 shows the nucleotide sequence of FAF-2 of 796 bp.

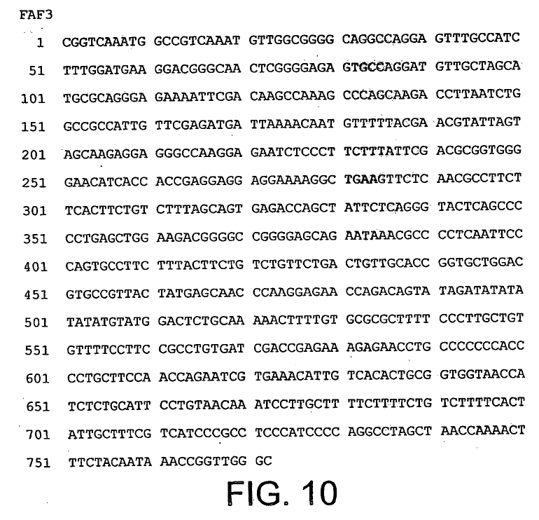

FIG. 10 shows the nucleotide sequence of FAF-3 of 772 bp.

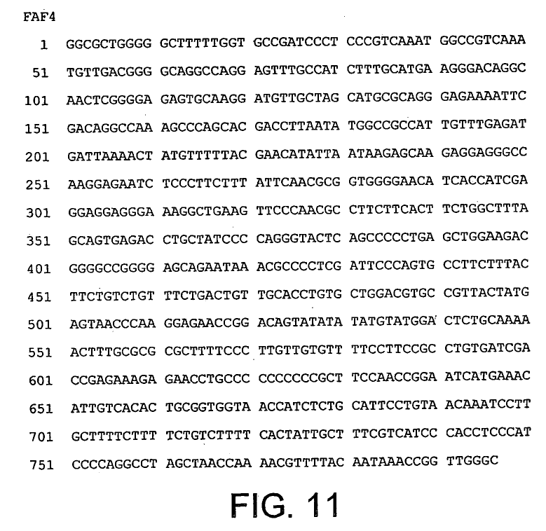

FIG. 11 shows the nucleotide sequence of FAF-4 of 796 bp.

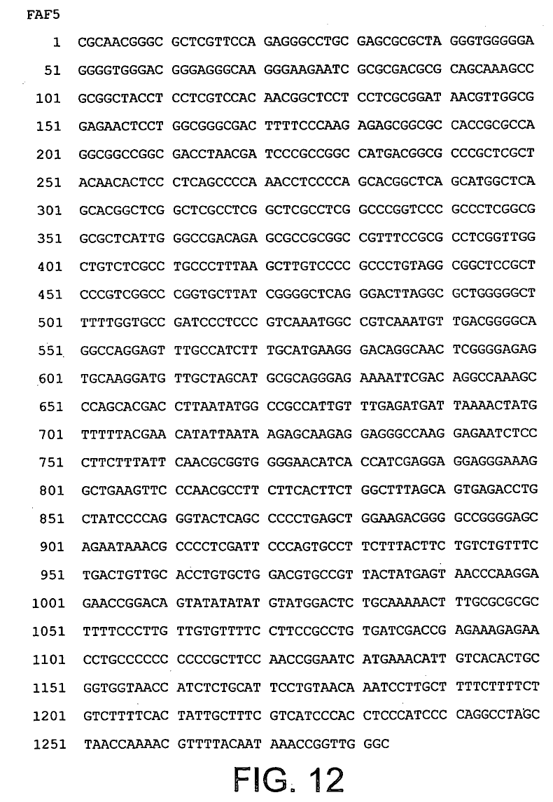

FIG. 12 shows the nucleotide sequence of FAF-5 of 1283 bp.

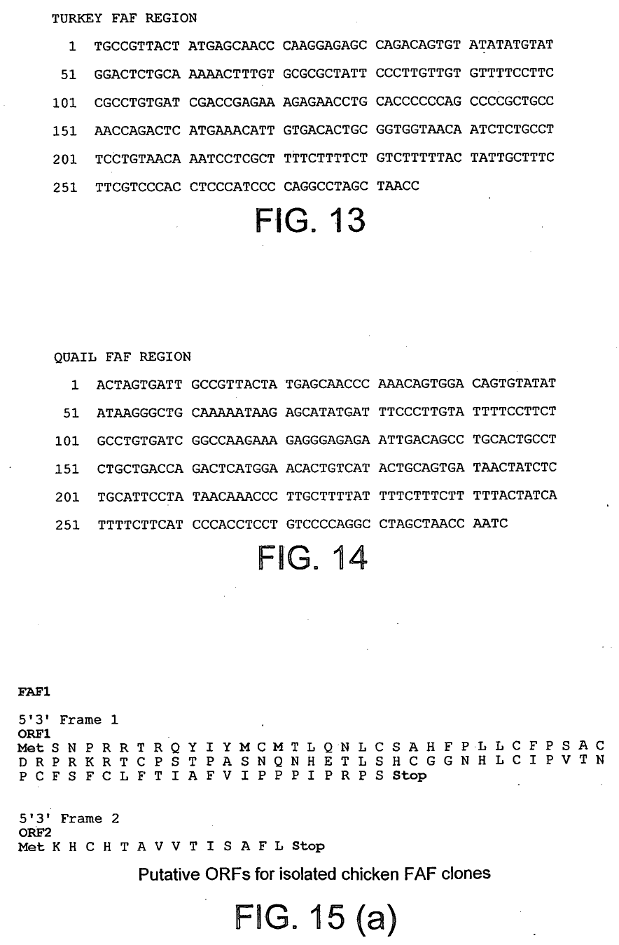

FIG. 13 shows a fragment of the nucleotide sequence of FAF from Turkey.

FIG. 14 shows a fragment of the nucleotide sequence of FAF from Quail.

FIG. 15 shows the putative ORFs for isolated chicken FAF clones: FIG. 15(a) shows the putative ORFs for FAF1, FIG. 15(b) for FAF2, FIG. 15(c) for FAF3, FIG. 15(d) for FAF4 and FIG. 15(e) for FAF5.

### EXAMPLE 1

### Differential Display Reverse Transcriptase—Polymerase Chain Reaction (DDRT-PCR)

DDRT-PCR is a powerful molecular tool which allows visualisation of gene expression in any particular cell type or tissue via the creation of RNA fingerprints. Genes which are differentially expressed between two or more samples under study are readily identifiable and recoverable using this technique. Bands representing differentially expressed cDNAs (for example, in male and female tissues) can be recovered and cloned (Miele et al In “Expression Genetics”, eds. McClelland & Pardee, pages 433-444, Natick: Eaton Publishing (1999)(a); Miele et al Prep. Biochem. Biotech. 29(3) 245-255 (1990)(b)). Cloned cDNAs are sequenced and identified following computer-assisted homology searching of the public nucleotide and protein databases. Cloned cDNAs were used for radiolabelling for use as probes in Northern and Southern hybridisation studies according to standard protocols.

Differential display analysis of RNA from male and female whole chicken embryos harvested on days 2.5, 3, 3.5, 4, and 4.5 using primers dT₁₂-MC (M=A,G,C) and DM8 (AGTGCCGTTA) revealed two bands which appeared to be female specific. These bands were cut out from the display gel, re-amplified using primers containing EcoR1 restriction sites and cloned into EcoR1 digested pB [symbol] SK⁺. Colonies obtained were screened for inserts, by PCR, using T7 and T3 primers. Two positive clones, 378.2.2 and 378.2.6 were obtained having insert sizes of approximately 350 bp, roughly the size of insert expected from the bands cut from the display gel. A fraction of the display reactions were run on an agarose/TBE gel and Southern blotted (Miele et al 2000). ³²P-labelled inserts from the isolated differential display clones were used to probe the blots. They gave the same female specific banding pattern, confirming that they corresponded to the cDNA bands cut from the display gel. Sequence analysis of the two clones revealed that they were identical.

Northern hybridisation is used to measure the amount and size of RNAs transcribed from eukaryotic genes. After isolating intact mRNA sequences, representing the products of gene transcription, the fragments can be separated and immobilised in a similar way to DNA sequences in Southern hybridisation. Major differences include the need for scrupulous handling to avoid degradation of the RNA by enzymes and the use of denaturing agents such as formamide to preserve the single-stranded, linear nature of the transcripts and allow them to be separated on the basis of their size. (Sambrook, J. & Russell, D. W., “Molecular Cloning: a Laboratory Manual” 3^{rd} edition, New York: Cold Spring Harbor Laboratory Press (2001)).

RNA samples from pooled male and pooled female whole chick embryos, days 2.5, 3, 3.5 and 4.5 were used to prepare northern blots. When differential display clone 378.2.2 was used as a probe, two major bands were detected in females. Transcript sizes were approximately 800 bp, 1300 bp. No bands were apparent in the male samples. The results are shown in FIG. 1.

### EXAMPLE 2

### Southern Hybridisation

Southern transfer is used to study how genes are organised within genomes using specific probes that hybridise to a portion of the gene. The genomic DNA is digested with restriction enzymes which cut at specific sites and produce a range of fragments of different sizes. The digested DNA is added to wells at one end of an agarose gel. Under the influence of an electric potential the DNA moves down the gel in columns, the fragments becoming separated by size, the smaller fragments moving more quickly. The DNA is transferred from the gel by blotting onto a solid support, such as a nylon membrane. This is then labelled with the radioactive probe which hybridises to the complementary sequences. These can be visualised as dark bands on a photographic negative in the process of autoradiography. (Sambrook, J. & Russell, D. W., “Molecular Cloning: a Laboratory Manual” 3^{rd} edition, New York: Cold Spring Harbor Laboratory Press (2001)).

A Southern blot of male and female chicken genomic DNA, digested with four different restriction enzymes, was probed with the ³²p labelled cDNA clone 378.2.6. A positive signal was seen in the female samples but no bands were detected in male samples even after the blots were overexposed. The results are shown in FIG. 2.

### EXAMPLE 3

### W-Specific PCR

PCR primers were designed from the 796 bp sequence of FAF-4. The sequences of the primers were FAF-Forward primer 5′-AGAATAAACGCCCCTCGATT-3′, and FAF reverse primer, 5′-CAGGTTCTCTTTCTCGGTCG-3′. PCR reactions were performed in 25 μl 10 mM Tris-HCl, 1.5 mM MgCl₂, 50 mM KCl pH8.3 containing 200 μM dNTP's, 0.8 μM primers and 1U Taq polymerase. Following an initial denaturation step of 2 minutes at 94° C., DNA was denatured at 94° C. for 30 seconds, annealed at 50° C., or 53° C., or 57° C. for 30 seconds and extended at 72° C. for 30 seconds. Reactions were subjected to 30 cycles of amplification. A final extension step at 72° C. for 5 minutes was carried out. After amplification, 20 μl of reaction mix was loaded onto a 1% TBE/agarose gel and electrophoresed for 1 hour at 100 volts. The results are shown in FIG. 3.

The PCR reaction described in this Example amplifies part of the conserved [324 bp]-nucleotide sequence of FAF which is present on the W-chromosome but not the Z-chromosome. FIG. 3 shows that for the three annealing temperatures in the region of the melting temperature of these two primers, there is amplification of the sequence in the female but not male samples. This specificity is maintained even at lower temperatures which increase the possibility of non-specific binding. The results demonstrate that the PCR method can successfully be applied to distinguish unambiguously between male and female DNA.

### EXAMPLE 4

### Analysis of Expression in Day 11 Chick Tissues

A northern blot of total RNA from male and female muscle, heart, brain, lung, liver, chorioallantoic membrane and mesonephrous was probed with ³²P-labelled clone 378.2.6. The female specific banding patterns obtained were identical in all tissues tested, differing only in the level of expression. The results are shown in FIG. 4.

### EXAMPLE 5

### Analysis of Location of FAF-4 796 bp Fragment

FIG. 5(a) shows the position of the [FAF]8 796 bp sequence in relation to the w-pkci gene and FIG. 5(b) shows the relative position of the PCR 204 bp product with respect to the [FAF]8 796 bp sequence clone. The forward primer (A) is

5′-AGAATAAACGCCCCTCGATT-3′

The reverse primer (B) is

5′-CAGGTTCTCTTTCTCGGTCG-3′

Primer Details:

| Oligo | Start | Length | Tm | GC % | Any | 3′ |
|---|---|---|---|---|---|---|
| Left primer | 414 | 20 | 59.93 | 45.00 | 4.00 | 2.00 |
| Right primer | 617 | 20 | 59.98 | 55.00 | 2.00 | 2.00 |

### EXAMPLE 6

### Species Blot

The results of a species blot probed with FAF display fragment are shown in FIG. 6. The samples probed were obtained from chicken, quail and turkey. Standard genomic DNA extraction from blood from these three species was followed by standard Southern analysis using the original 324 bp FAF-1 fragment as a probe.

### EXAMPLE 7

### Sampling of Amnion and Allantois of a Developing Chick Embryo

In order to perform a method of the invention for the purposes of determining the sex of a chick embryo, it is necessary to obtain samples of the amnion and/or allantois of the embryo inside the egg. Small volumes (5 μl to 25 μl) of amniotic and allantoic fluid can be removed manually or by automated sampling. The collected fluids can then be used as substrates in chick-sexing PCR methods according to the present invention. Sampling of fluids from the chick embryo is shown diagrammatically in FIG. 7.

### EXAMPLE 8

### Sequencing of Differentially Expressed RNAs

The sequence of the insert of approximately 350 bp found in the two positive clones, 378.2.2 and 378.2.6 identified in Example 1 was sequenced by the method of dideoxy chain termination analysis. The sequence of the FAF-display fragment is shown in FIG. 8.

The DNA sequences corresponding to the approximately 800 bp and 1300 bp RNA bands identified in Example 1 were sequenced as above. The results are shown in FIGS. 9 to 12 as sequences FAF-2, FAF-3, FAF-4 and FAF-5.

The sequence information obtained was subjected to further analysis to look for homology with other sequences in the available databases and to compare the different sequences with each other.

The differential display fragment of 350 bp was found not to match exactly with any of FAF-2, FAF-3, FAF-4, or FAF-5, but a considerable degree of overlap was seen with only relatively few base pair substitutions, or gaps.

In the comparison of the other sequences, FAF-2 was found to show only a 2 nucleotide difference from FAF-5 and FAF-4 when the sequences were aligned. Sequence FAF-2 has a 90% homology with sequence FAF-3 and sequence FAF-4 matches FAF-5 exactly over 796 nucleotides.

In a BLAST 2.2.1 search on www.ncbi.nlm.nih.gov, it was found that FAF-4 has four nucleotide differences with the 5′ genonic non-translated region of the wpcki gene. The search as a BLAST search of the nr (non-redundant) nucleotide databases.

It is concluded that FAF is located on the complementary strand of the wpkci repeat region, in which there are approximately 40 repeats of the wpcki gene. However, it lies in the inter-genic region of those repeats with less well conserved sequences. Four FAF transcripts have now been identified and slight differences in the sequence (or in the case of FAF-4 and FAF-5, the different lengths) suggests that they each come from a different repeat of the gene.

### EXAMPLE 9

### Predicted Protein Coding Sequences and Antibodies

Analysis of the FAF sequences shows that open-reading frames encoding proteins exist. The FAF peptides of the ORFs are shown in FIG. 15.

The FAF sequences are expressed in a suitable host cell, typically an avian in vitro culture system such as the method of Perry et al (EP-A-0295964) and purified using affinity chromatography. Purified FAF peptides, fragments or fusion proteins thereof can be used to generate monoclonal antibodies against FAF using conventional techniques, for example those described in Antibodies: A Laboratory Manual, Harlow and Lane, eds. Cold Spring Harbor Laboratory Press (1988)).

Briefly, mice are immunised with a FAF peptide as an immunogen emulsified in complete Freund's adjuvant, and injected in amounts ranging from 10-100 μg subcutaneously or intraperitoneally. Ten to twelve days later, the immunised animals are boosted with additional FAF peptide emulsified in incomplete Freund's adjuvant. Mice are periodically boosted thereafter on a weekly to bi-weekly immunisation schedule. Serum samples are periodically taken by retro-orbital bleeding or tail-tip excision to test for anti-FAF peptide antibodies by dot blot assay, or ELISA.

Following detection of an appropriate antibody titre, positive animals are provided one last intravenous injection of FAF peptide in saline. Three to four days later, the animals are sacrificed, spleen cells harvested, and spleen cells are fused to a murine myeloma cell line, e.g. NS1 or preferably P3x63Ag8.653 (ATCC CRL 1580). Fusions generate hybridoma cells, which are plated in multiple microtitre plates in a HAT (hypoxanthine, aminopterin and thymidine) selective medium to inhibit proliferation of non-fused cells, myeloma hybrids and spleen cell hybrids.

The hybridoma cells are screened by ELISA for reactivity against purified FAF peptides by adaptations of the techniques described in Engvall et al (Immunochem. 8, 871 (1971)) or Beckmann et al (J. Immunol. 144, 4212 (1990)). Positive hybridoma cells can be injected into syngeneic BALB/c mice to produce ascites containing high concentrations of anti-FAF monoclonal antibodies. Alternatively, hybridoma cells can be grown in vitro in flasks or roller bottles by various techniques. Monoclonal antibodies produced in mouse ascites can be purified by ammonium sulphate precipitation, followed by gel exclusion chromatography. Alternatively, affinity chromatography based upon binding of antibody to protein A or protein G can also be used, as can affinity chromatography based upon binding to FAF peptides. The resultant antibodies may be suitably stored in a physiological solution, such as phosphate buffered saline.

1.-20. (canceled)

21. A method for the determination of the sex of an avian subject, the method comprising contacting a sample from said subject with a nucleic acid probe comprising an at least 6 base pair fragment from a target nucleic acid sequence FAF-4 as shown in FIG. 11, or a sequence complementary or homologous thereto.

22. A method as claimed in claim 21, in which the nucleic acid probe comprises a probe sequence of at least 15 nucleotides.

23. A method as claimed in claim 21, in which the nucleic acid probe is sequence FAF-4 as shown in FIG. 11 or a fragment thereof.

24. A method as claimed in claim 21, in which the avian is a member of Class Aves.

25. A method as claimed in claim 24, in which the avian is selected from the group consisting of Gallus gallus (chicken), turkey, quail, and guinea fowl.

26. A method as claimed in claim 21, in which the sample is allantoic fluid or amniotic fluid.

27. A method as claimed in claim 21, in which the sample is taken from an egg.

28. A method as claimed in claim 21, in which the analysis of the sample comprises a nucleic acid amplification procedure.

29. A method as claimed in claim 28, in which the nucleic acid amplification procedure is exponential amplification of the target sequence.

30. A method as claimed in claim 29, in which the nucleic acid amplification procedure is linear amplification of the target sequence.

31. A method as claimed in claim 30, which comprises amplification of RNA in the sample.

32. An isolated nucleic acid molecule as shown in FIG. 11.

33. A kit of parts comprising a nucleic acid probe comprising an at least 6 base pair fragment from an isolated nucleic acid molecule FAF-4 as shown in FIG. 11 for determining the sex of an avian subject, or a sequence complementary or homologous thereto.

34. A polypeptide or fragment thereof coded for by a nucleic acid sequence of claim 32.

35. A polypeptide or fragment thereof as claimed in claim 34, wherein the sequence comprises a sequence as shown in FIG. 15(d).

36. A vector comprising a nucleic acid sequence of claim 32.

37. A host cell comprising a vector as defined in claim 36.

38. An antibody to a polypeptide as defined in claim 34.

39. An antibody as claimed in claim 38, which is a monoclonal antibody.

40. A method for the determination of the sex of an avian subject, the method comprising contacting a sample from said subject with an antibody to a polypeptide as defined in claim 34.

41. A kit of parts comprising an antibody as defined in claim 38 for determining the sex of an avian subject.


SEQUENCE LISTING (30 sequences)

SEQ ID NO: 1 - DNA, 318 bp, Gallus gallus
agtgccgttactatgagcaacccaaggagaaccagacagtatatatatatgtgtatgactctgcaaaacctttgtagcgcgcattttcccttgctgtgttttccttccgcctgtgatcgaccgagaaagagaacctgcccctctacccctgcttccaaccagaatcatgaaacactgtcacactgcggtggtaaccatctctgcattcctgtaacaaatccttgcttttctttctgtctttttactattgctttcgtcatcccacctcccatcccccggcctagctaaccaaaactttctacaataaaccggttgggc

SEQ ID NO: 2 - DNA, 796 bp, Gallus gallus
ggcgctgggggctttttggtgccgatccctcccgtcaaatggccgtcaaatgttgacggggcaggccaggagtttgccatctttgcatgaagggacaggcaactcggggagagtgcaaggatgttgctagcatgcgcagggagaaaattcgacaggccaaagcccagcacgaccttaatatggccgccattgtttgagatgattaaaactatgtttttacgaacatattaataagagcaagaggagggccaaggagaatctcccttctttattcaacgcggtggggaacatcaccatcgaggaggagggaaaggctgaagttcccaacgccttcttcacttctggctttagcagtgagacctgctatccccagggtactcagccccctgagctggaagacggggccggggagcagaataaacgcccctcgattcccagtgccttctttacttctgtctgtttctgactgttgcacctgtgctggacgtgccgttactatgagtaacccaaggagaaccggacagtatatatatgtatggaccctgcaaaaactttgcgcgcgcttttcccttgttgtgttttccttccgcctgtgatcgaccgagaaagagaacccgccccccccccgcttccaaccggaatcatgaaacattgtcacactgcggtggtaaccatctctgcattcctgtaacaaatccttgcttttcttttctgtcttttcactattgctttcgtcatcccacctcccatccccaggcctagctaaccaaaacgttttacaataaaccggttgggc

SEQ ID NO: 3 - DNA, 772 bp, Gallus gallus
cggtcaaatggccgtcaaatgttggcggggcaggccaggagtttgccatctttggatgaaggacgggcaactcggggagagtgccaggatgttgctagcatgcgcagggagaaaattcgacaagccaaagcccagcaagaccttaatctggccgccattgttcgagatgattaaaacaatgtttttacgaacgtattagtagcaagaggagggccaaggagaatctcccttctttattcgacgcggtggggaacatcaccaccgaggaggaggaaaaggctgaagttctcaacgccttcttcacttctgtctttagcagtgagaccagctattctcagggtactcagccccctgagctggaagacggggccggggagcagaataaacgcccctcaattcccagtgccttctttacttctgtctgttctgactgttgcaccggtgctggacgtgccgttactatgagcaacccaaggagaaccagacagtatagatatatatatatgtatggactctgcaaaaacttttgtgcgcgcttttcccttgctgtgttttccttccgcctgtgatcgaccgagaaagagaacctgcccccccacccctgcttccaaccagaatcgtgaaacattgtcacactgcggtggtaaccatctctgcattcctgtaacaaatccttgcttttcttttctgtcttttcactattgctttcgtcatcccgcctcccatccccaggcctagctaaccaaaactttctacaataaaccggttgggc

SEQ ID NO: 4 - DNA, 796 bp, Gallus gallus
ggcgctgggggctttttggtgccgatccctcccgtcaaatggccgtcaaatgttgacggggcaggccaggagtttgccatctttgcatgaagggacaggcaactcggggagagtgcaaggatgttgctagcatgcgcagggagaaaattcgacaggccaaagcccagcacgaccttaatatggccgccattgtttgagatgattaaaactatgtttttacgaacatattaataagagcaagaggagggccaaggagaatctcccttctttattcaacgcggtggggaacatcaccatcgaggaggagggaaaggctgaagttcccaacgccttcttcacttctggctttagcagtgagacctgctatccccagggtactcagccccctgagctggaagacggggccggggagcagaataaacgcccctcgattcccagtgccttctttacttctgtctgtttctgactgttgcacctgtgctggacgtgccgttactatgagtaacccaaggagaaccggacagtatatatatgtatggactctgcaaaaactttgcgcgcgcttttcccttgttgtgttttccttccgcctgtgatcgaccgagaaagagaacctgccccccccccgcttccaaccggaatcatgaaacattgtcacactgcggtggtaaccatctctgcattcctgtaacaaatccttgcttttcttttctgtcttttcactattgctttcgtcatcccacctcccatccccaggcctagctaaccaaaacgttttacaataaaccggttgggc

SEQ ID NO: 5 - DNA, 1283 bp, Gallus gallus
cgcaacgggcgctcgttccagagggcctgcgagcgcgctagggtgggggaggggtgggacgggagggcaagggaagaatcgcgcgacgcgcagcaaagccgcggctacctcctcgtccacaacggctcctcctcgcggataacgttggcggagaactcctggcgggcgacttttcccaagagagcggcgccaccgcgccaggcggccggcgacctaacgatcccgccggccatgacggcgcccgctcgctacaacactccctcagccccaaacctccccagcacggctcagcatggctcagcacggctcggctcgcctcggctcgcctcggcccggtcccgccctcggcggcgctcattgggccgacagagcgccgcggccgtttccgcgcctcggttggctgtctcgcctgccctttaagcttgtccccgccctgtaggcggctccgctcccgtcggcccggtgcttatcggggctcagggacttaggcgctgggggctttttggtgccgatccctcccgtcaaatggccgtcaaatgttgacggggcaggccaggagtttgccatctttgcatgaagggacaggcaactcggggagagtgcaaggatgttgctagcatgcgcagggagaaaattcgacaggccaaagcccagcacgaccttaatatggccgccattgtttgagatgattaaaactatgtttttacgaacatattaataagagcaagaggagggccaaggagaatctcccttctttattcaacgcggtggggaacatcaccatcgaggaggagggaaaggctgaagttcccaacgccttcttcacttctggctttagcagtgagacctgctatccccagggtactcagccccctgagctggaagacggggccggggagcagaataaacgcccctcgattcccagtgccttctttacttctgtctgtttctgactgttgcacctgtgctggacgtgccgttactatgagtaacccaaggagaaccggacagtatatatatgtatggactctgcaaaaactttgcgcgcgcttttcccttgttgtgttttccttccgcctgtgatcgaccgagaaagagaacctgccccccccccgcttccaaccggaatcatgaaacattgtcacactgcggtggtaaccatctctgcattcctgtaacaaatccttgcttttcttttctgtcttttcactattgctttcgtcatcccacctcccatccccaggcctagctaaccaaaacgttttacaataaaccggttgggc

SEQ ID NO: 6 - DNA, 285 bp, Meleagris gallopavo
tgccgttactatgagcaacccaaggagagccagacagtgtatatatgtatggactctgcaaaaactttgtgcgcgctattcccttgttgtgttttccttccgcctgtgatcgaccgagaaagagaacctgcaccccccagccccgctgccaaccagactcatgaaacattgtgacactgcggtggtaacaatctctgccttcctgtaacaaatcctcgcttttcttttctgtctttttactattgctttcttcgtcccacctcccatccccaggcctagctaacc

SEQ ID NO: 7 - DNA, 294 bp, Coturnix coturnix
actagtgattgccgttactatgagcaacccaaacagtggacagtgtatatataagggctgcaaaaataagagcatatgatttcccttgtattttccttctgcctgtgatcggccaagaaagagggagagaattgacagcctgcactgcctctgctgaccagactcatggaacactgtcatactgcagtgataactatctctgcattcctataacaaacccttgcttttattttctttctttttactatcattttcttcatcccacctcctgtccccaggcctagctaaccaatc

SEQ ID NO: 8 - protein, 91 aa, Gallus gallus
MSNPRRTRQYIYMCMTLQNLCSAHFPLLCFPSACDRPRKRTCPSTPASNQNHETLSHCGGNHLCIPVTNPCFSFCLFTIAFVIPPPIPRPS

SEQ ID NO: 9 - protein, 15 aa, Gallus gallus
MKHCHTAVVTISAFL

SEQ ID NO: 10 - protein, 36 aa, Gallus gallus
MLLACAGRKFDRPKPSTTLIWPPLFEMIKTMFLRTY

SEQ ID NO: 11 - protein, 76 aa, Gallus gallus
MYGPCKNFARAFPLLCFPSACDRPRKRTRPPPASNRNHETLSHCGGNHLCIPVTNPCFSFLSFHYCFRHPTSHPQA

SEQ ID NO: 12 - protein, 26 aa, Gallus gallus
MLTGQARSLPSLHEGTGNSGRVQGCC

SEQ ID NO: 13 - protein, 51 aa, Gallus gallus
MDPAKTLRALFPCCVFLPPVIDREREPAPPPLPTGIMKHCHTAVVTISAFL

SEQ ID NO: 14 - protein, 5 aa, Gallus gallus
MAVKC

SEQ ID NO: 15 - protein, 36 aa, Gallus gallus
MKGQATRGECKDVASMRREKIRQAKAQHDLNMAAIV

SEQ ID NO: 16 - protein, 32 aa, Gallus gallus
MSNPRRTGQYIYVWTLQKLCARFSLVVFSFRL

SEQ ID NO: 17 - protein, 51 aa, Gallus gallus
MLAGQARSLPSLDEGRATRGECQDVASMRREKIRQAKAQQDLNLAAIVRDD

SEQ ID NO: 18 - protein, 76 aa, Gallus gallus
MDSAKTFVRAFPLLCFPSACDRPRKRTCPPTPASNQNRETLSHCGGNHLCIPVTNPCFSFLSFHYCFRHPASHPQA

SEQ ID NO: 19 - protein, 63 aa, Gallus gallus
MAVKCWRGRPGVCHLWMKDGQLGESARMLLACAGRKFDKPKPSKTLIWPPLFEMIKTMFLRTY

SEQ ID NO: 20 - protein, 35 aa, Gallus gallus
MSNPRRTRQYRYIYMYGLCKNFCARFSLAVFSFRL

SEQ ID NO: 21 - protein, 76 aa, Gallus gallus
MYGLCKNFARAFPLLCFPSACDRPRKRTCPPPASNRNHETLSHCGGNHLCIPVTNPCFSFLSFHYCFRHPTSHPQA

SEQ ID NO: 22 - protein, 51 aa, Gallus gallus
MDSAKTLRALFPCCVFLPPVIDREREPAPPPLPTGIMKHCHTAVVTISAFL

SEQ ID NO: 23 - protein, 62 aa, Gallus gallus
MTAPARYNTPSAPNLPSTAQHGSARLGSPRLASARSRPRRRSLGRQSAAAVSAPRLAVSPAL

SEQ ID NO: 24 - protein, 48 aa, Gallus gallus
MAQHGSARLGSPRPGPALGGAHWADRAPRPFPRLGWLSRLPFKLVPAL

SEQ ID NO: 25 - DNA, 14 bp, Artificial sequence
  Description of Artificial Sequence Synthetic primer
ttttttttttttvc

SEQ ID NO: 26 - DNA, 10 bp, Artificial sequence
  Description of Artificial Sequence Synthetic primer
agtgccgtta

SEQ ID NO: 27 - DNA, 20 bp, Artificial sequence
  Description of Artificial Sequence Synthetic primer
agaataaacgcccctcgatt

SEQ ID NO: 28 - DNA, 20 bp, Artificial sequence
  Description of Artificial Sequence Synthetic primer
caggttctctttctcggtcg

SEQ ID NO: 29 - DNA, 20 bp, Artificial sequence
  Description of Artificial Sequence Synthetic primer
agaataaacgcccctcgatt

SEQ ID NO: 30 - DNA, 20 bp, Artificial sequence
  Description of Artificial Sequence Synthetic primer
caggttctctttctcggtcg